(19) United States
(12) Patent Application Publication
(10) Pub. No.: US 2023/0172427 A1
(43) Pub. Date: Jun. 8, 2023

(54) METHODS AND RELATED ASPECTS FOR EAR PATHOLOGY DETECTION

(71) Applicant: THE JOHNS HOPKINS UNIVERSITY, Baltimore, MD (US)

(72) Inventors: James Henri CLARK, Owings Mills, MD (US); Therese L. CANARES, Baltimore, MD (US); Mathias UNBERATH, Baltimore, MD (US); John Robertson RZASA, University Park, MD (US)

(73) Assignee: THE JOHNS HOPKINS UNIVERSITY, Baltimore, MD (US)

(21) Appl. No.: 17/995,455

(22) PCT Filed: Apr. 5, 2021
(86) PCT No.: PCT/US2021/025770

§ 371 Date: Oct. 4, 2022

Related U.S. Application Data
(60) Provisional application No. 63/007,641, filed on Apr. 9, 2020.

(51) Int. Cl. A61B 1/00; A61B 1/227; G16H 50/70

(52) U.S. Cl. CPC A61B 1/000096; A61B 1/00052; A61B 1/227; G16H 50/70

(57) ABSTRACT

Provided herein are methods of detecting a pathology in an ear of a subject that include matching properties of captured images and/or videos with properties of an ear pathology model that is trained on a plurality of reference images and/or videos of ears of reference subjects, which properties of the ear pathology model are indicative of the pathology. Related kits, devices, systems, and computer program products are also provided.

### CROSS-REFERENCE TO RELATED APPLICATIONS

This application claims priority to, and the benefit of, U.S. Provisional Patent Application Ser. No. 63/007,641, filed Apr. 9, 2020, the disclosure of which is incorporated herein by reference.

### BACKGROUND

Ear infections place a significant burden on the healthcare system, with nearly nine million ear infections diagnosed in the US annually. Ear complaints are the leading presenting complaint in pediatric patients seeking medical attention and being prescribed an antibiotic [Soni A. Statistical Brief: #228. Ear Infections (Otitis Media) in Children (0-17): Use and Expenditures, 2006. Medical Expenditure Panel Survey. 2008:1-5]. The majority of patients diagnosed with an ear infection by a healthcare provider are discharged home with an antibiotic prescription and outpatient follow up. Despite the low medical complexity of these encounters, on average it takes about three hours from registration to discharge in an emergency department. The length of the encounter is typically related to the antiquated diagnostic process. The modern process and otoscope would likely be readily recognized by a clinician who trained over a century ago.

Using the standard clinical otoscope consisting of a light source and a 15 mm eyepiece with 3× magnification, a medically trained healthcare provider (e.g., physician (M.D.), nurse practitioner (N.P.), or physician assistant (P.A.)) is needed to see the tympanic membrane and to make the diagnostic determination. Once visualized, the determination regarding the presence or absence of disease is generally based on the healthcare provider's previous experience. This diagnosis process is often further complicated by suboptimal visualization of the ear canal and tympanic membrane due, for example, to patient movements during the examination (especially when the patients are young children), the presence of ear wax obstructions in the patient's ear canal, and/or limited otoscope magnification, among other factors.

Not surprisingly, studies have shown that clinicians only correctly diagnose ear infections at marginally higher accuracy rates (53%) than if the diagnosis were based on a mere coin toss [Buchanan et al., “Recognition of paediatric otopathology by General Practitioners,” International Journal of Pediatric Otorhinolaryngology, 72(5):669-673 (2008)]. The uncertainty in the diagnostic accuracy often leads to a corresponding compensatory over-prescription of antibiotics. To illustrate, it is estimated that up to a quarter of antibiotics prescribed for ear infections are unnecessary [Rosenfeld, “Diagnostic certainty for acute otitis media,” Int J Pediatr Otorhinolaryngol, 64(2):89-95 (2002)]. Further, the U.S. spends nearly $3 billion annually on the management of ear infections. As a result, eliminating unnecessary antibiotic prescriptions could yield a $300 million savings per year [Id.].

Accordingly, there is a need for additional methods, and related aspects, for diagnosing otologic pathologies.

### SUMMARY

The present disclosure relates, in certain aspects, to methods, devices, kits, systems, and computer readable media of use in detecting ear pathologies. In certain applications, for example, the smart otoscope devices disclosed herein capture images and/or videos of the ear canal and/or tympanic membrane of the ear of a given subject, display those images and/or videos, and match properties (e.g., patterns or the like) of the captured images and/or videos with properties of an ear pathology model that is trained on a plurality of reference images and/or videos of ear canals and/or tympanic membranes of ears of reference subjects. The properties of the ear pathology model are indicative of at least one pathology. These and other aspects will be apparent upon a complete review of the present disclosure, including the accompanying figures.

In certain aspects, the present disclosure provides a method of detecting a pathology in an ear of a subject. The method includes capturing (e.g., magnifying and recording, etc.), by an otoscope, one or more images and/or videos of an ear canal and/or tympanic membrane of the ear of the subject, wherein the otoscope comprises at least one camera to generate at least one captured image. The method also includes matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of the pathology, thereby detecting the pathology in the ear of the subject. In some embodiments, the camera comprises one or more microscopes and/or miniature cameras (e.g., a small image sensor having a wide field-of-view (FOV) and a short working distance, or the like). In certain embodiments, the properties comprise one or more patterns. In some embodiments, the ear pathology model is generated using one or more machine learning algorithms. In some of these embodiments, the machine learning algorithms comprise one or more neural networks. In certain embodiments, the capturing and matching steps are performed substantially in real-time. In some embodiments, the method includes using hyperspectral imaging and/or optical coherence tomography (OCT) to capture the images and/or videos of the ear canal and/or tympanic membrane of the ear of the subject. In some of these embodiments, the method includes illuminating the ear canal and/or tympanic membrane of the ear of the subject with many different wavelengths, measuring wavelength resolved signal, and performing Fourier transform on the resolved signal (e.g., time domain OCT).

In certain embodiments, the pathology comprises one or more of: otitis media, otitis media with effusion, mucoid otits media, otosclerosis, cholesteatoma, direct trauma, infected furuncle, necrotizing otitis externa, herpes zoster, acquired stenosis, osteoma, acute otitis externa, chronic otitis externa, deep impacted wax, retraction pocket, keratosis obturans, granular myringitis, bullous myringitis, tympanosclerosis, perforation, tympanosclerosis, glomus tumour, posterior infection, hemotympanum, foreign body, and temporal bone fracture.

In some embodiments, the method further includes administering one or more therapies to the subject to treat the pathology. In certain embodiments, the method further includes repeating the method at one or more later time points to monitor progression of the pathology in the subject. In some embodiments, the ear pathology model comprises one or more selected therapies indexed to the pathology in the ear of the subject.

In certain embodiments, the otoscope comprises one or more working ports or channels and the method further comprises inserting one or more implements through the working ports or channels into the ear canal of the subject to clean, retrieve foreign bodies from, and/or take samples from the ear canal and/or the tympanic membrane of the ear of the subject when the otoscope captures the images and/or videos of the ear canal and/or the tympanic membrane of the ear of the subject. In some embodiments, the otoscope comprises at least one display screen operably connected to the camera and the method further comprises displaying the images and/or videos on the display screen when the otoscope captures the images and/or videos of the ear canal and/or the tympanic membrane of the ear of the subject.

In some embodiments, the otoscope is operably connected to a database comprising an electronic medical record (EMR) of the subject and wherein the method further comprises retrieving data from the electronic medical record and/or populating the electronic medical record with at least one of the images and/or videos, and/or information related thereto. In certain of these embodiments, the otoscope is wirelessly connected, or connectable, to the electronic medical record of the subject. In certain embodiments, the otoscope device and/or the database is wirelessly connected, or connectable, to one or more communication devices of one or more remote users and wherein the remote users view at least one of the images and/or videos of the canal and/or tympanic membrane of the ear of the subject and/or the electronic medical record of the subject using the communication devices. In certain of these embodiments, the communication devices comprise one or more mobile applications that operably interface with the otoscope device and/or the database. In some of these embodiments, the users input one or more entries into the electronic medical record of the subject in view of the detected pathology in the ear of the subject using the communication devices. In certain of these embodiments, the users order one or more therapies and/or additional analyses of the subject in view of the detected pathology in the ear of the subject using the communication devices. In some of these embodiments, a system that comprises the database automatically orders one or more therapies and/or additional analyses of the subject in view of the detected pathology in the ear of the subject when the users input the entries into the electronic medical record of the subject.

In some embodiments, the otoscope comprises at least one illumination source and the method further comprises illuminating the ear canal and/or tympanic membrane of the ear of the subject using the illumination source when capturing the images and/or videos of the ear canal and/or tympanic membrane of the ear of the subject. In certain of these embodiments, the illumination source is configured to illuminate at two or more selectable illumination wavelengths and wherein the method further comprises selecting at least one of the selectable illumination wavelengths prior to or when capturing the images and/or videos of the ear canal and/or tympanic membrane of the ear of the subject. In certain of these embodiments, the selectable illumination wavelengths comprise at least one visible wavelength and/or at least one infrared wavelength. In some of these embodiments, the illumination source is configured to illuminate in one or more selectable illumination modes and wherein the method further comprises selecting at least one of the selectable illumination modes prior to or when capturing the images and/or videos of the ear canal and/or tympanic membrane of the ear of the subject. In certain of these embodiments, the selectable illumination modes comprise at least one pulsed illumination mode.

In some aspects, the present disclosure provides a method of treating a pathology in an ear of a subject. The method includes capturing, by an otoscope, one or more images and/or videos of an ear canal and/or tympanic membrane of the ear of the subject, wherein the otoscope comprises at least one camera to generate at least one captured image. The method also includes matching one or more properties of the captured image with one or more properties of at least one ear pathology model that is trained on a plurality of reference images and/or videos of ear canals and/or tympanic membranes of ears of reference subjects, which properties of the ear pathology model are indicative of the pathology to thereby detect the pathology in the ear of the subject. In addition, the method also includes administering one or more therapies to the subject, thereby treating the pathology in the ear of the subject.

In some aspects, the present disclosure provides an otoscope device that includes a body structure, at least one speculum operably connected to the body structure, at least one camera at least partially disposed within the speculum, which camera is configured to capture one or more images and/or videos of an ear canal and/or tympanic membrane of an ear of a subject when the speculum is disposed at least proximal to the ear canal and/or tympanic membrane of the ear of the subject, and at least one display screen operably connected to the body structure, which display screen is configured to display the images and/or videos of the ear canal and/or tympanic membrane of the ear of the subject when the speculum is disposed at least proximal to the ear canal and/or tympanic membrane of the ear of the subject. The otoscope device also includes at least one controller at least partially disposed within the body structure, which controller is operably connected at least to the camera and to the display screen, wherein the controller comprises, or is capable of accessing, computer readable media comprising non-transitory computer executable instructions which, when executed by at least one electronic processor, perform at least: capturing the images and/or videos of the ear canal and/or tympanic membrane of the ear of the subject when the speculum is disposed at least proximal to the ear canal and/or tympanic membrane of the ear of the subject; displaying the captured images and/or videos of the ear canal and/or tympanic membrane of the ear of the subject when the speculum is disposed at least proximal to the ear canal and/or tympanic membrane of the ear of the subject; and matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of at least one pathology. In addition, the device otoscope device also includes at least one power source operably connected, or connectable, to one or more of the controller, the camera, and the display screen.

In some embodiments, a kit includes the otoscope device. In certain embodiments, the camera comprises one or more microscopes and/or miniature cameras. In certain embodiments, the properties comprise one or more patterns. In certain embodiments, the ear pathology model is generated using one or more machine learning algorithms. In some of these embodiments, the machine learning algorithms comprise one or more neural networks. In certain embodiments, the ear pathology model comprises one or more selected therapies indexed to the pathology.

In some embodiments, the body structure and/or the speculum comprises one or more working ports or channels through which one or more implements are inserted into the ear canal of the subject to clean, retrieve foreign bodies from, and/or take samples from the ear canal and/or the tympanic membrane of the ear of the subject when the otoscope captures the images and/or videos of the ear canal and/or the tympanic membrane of the ear of the subject. In certain embodiments, the controller is wirelessly connected, or connectable, to one or more of the computer executable instructions.

In certain embodiments, the controller is operably connected, or connectable, to a database comprising an electronic medical record of the subject and wherein the computer executable instructions further perform retrieving data from the electronic medical record and/or populating the electronic medical record with at least one of the images and/or videos, and/or information related thereto. In some of these embodiments, the controller is wirelessly connected, or connectable, to the electronic medical record of the subject. In certain of these embodiments, the otoscope device and/or the database is wirelessly connected, or connectable, to one or more communication devices of one or more remote users and wherein the remote users view at least one of the images and/or videos of the canal and/or tympanic membrane of the ear of the subject and/or the electronic medical record of the subject using the communication devices. In some of these embodiments, the communication devices comprise one or more mobile applications that operably interface with the otoscope device and/or the database. In some of these embodiments, the users are capable of inputting one or more entries into the electronic medical record of the subject in view of the detected pathology in the ear of the subject using the communication devices. In certain of these embodiments, the users are capable of ordering one or more therapies and/or additional analyses of the subject in view of the detected pathology in the ear of the subject using the communication devices.

In certain embodiments, the otoscope device further comprises at least one illumination source operably connected to the controller, which illumination source is configured to illuminate the ear canal and/or tympanic membrane of an ear of the subject when the speculum is disposed at least proximal to the ear canal and/or tympanic membrane of the ear of the subject. In some of these embodiments, the illumination source is configured to illuminate at two or more selectable illumination wavelengths and wherein the computer executable instructions further perform at least: causing the illumination source to illuminate at a selected illumination wavelength in response to a user selection. In certain of these embodiments, the selectable illumination wavelengths comprise at least one visible wavelength and/or at least one infrared wavelength. In some of these embodiments, the illumination source is configured to illuminate in one or more selectable illumination modes and wherein the computer executable instructions further perform at least: causing the illumination source to illuminate at a selected illumination mode in response to a user selection. In certain of these embodiments, the selectable illumination modes comprise at least one pulsed illumination mode. In some embodiments, the controller is configured to capture the images and/or videos of the ear canal and/or tympanic membrane of the ear of the subject using hyperspectral imaging and/or optical coherence tomography (OCT).

In some aspects, the present disclosure provides a system that includes at least one otoscope device that comprises at least one camera that is configured to capture one or more images and/or videos of an ear canal and/or tympanic membrane of an ear of a subject when the camera is disposed at least proximal to the ear canal and/or tympanic membrane of the ear of the subject. The system also includes at least one controller that is operably connected, or connectable, at least to the camera, wherein the controller comprises, or is capable of accessing, computer readable media comprising non-transitory computer executable instructions which, when executed by at least one electronic processor, perform at least: capturing the images and/or videos of the ear canal and/or tympanic membrane of the ear of the subject when the speculum is disposed at least proximal to the ear canal and/or tympanic membrane of the ear of the subject; and matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of at least one pathology.

In some aspects, the present disclosure provides a computer readable media (e.g., embodying a diagnostic AI-based algorithm) comprising non-transitory computer executable instruction which, when executed by at least electronic processor perform at least: capturing, by an otoscope, one or more images and/or videos of an ear canal and/or tympanic membrane of the ear of a subject, wherein the otoscope comprises at least one camera to generate at least one captured image; and matching one or more properties of the captured image with one or more properties of at least one ear pathology model that is trained on a plurality of reference images and/or videos of ear canals and/or tympanic membranes of ears of reference subjects, which properties of the ear pathology model are indicative of the pathology, thereby detecting the pathology in the ear of the subject.

### BRIEF DESCRIPTION OF THE DRAWINGS

The accompanying drawings, which are incorporated in and constitute a part of this specification, illustrate certain embodiments, and together with the written description, serve to explain certain principles of the methods, devices, kits, systems, and related computer readable media disclosed herein. The description provided herein is better understood when read in conjunction with the accompanying drawings which are included by way of example and not by way of limitation. It will be understood that like reference numerals identify like components throughout the drawings, unless the context indicates otherwise. It will also be understood that some or all of the figures may be schematic representations for purposes of illustration and do not necessarily depict the actual relative sizes or locations of the elements shown.

FIG. 1A schematically depicts an otoscope device from a perspective view according to one exemplary embodiment.

FIG. 1B schematically depicts the otoscope device of FIG. 1A from another perspective view.

FIG. 2A schematically depicts an otoscope device from a perspective view according to one exemplary embodiment.

FIG. 2B schematically depicts the otoscope device of FIG. 2A from another perspective view.

FIG. 2C schematically depicts the otoscope device of FIG. 2A from a side view.

FIG. 3 is a schematic diagram that depicts exemplary otoscope device components according to some aspects disclosed herein.

FIG. 4 is a flow chart that schematically depicts exemplary method steps according to some aspects disclosed herein.

FIG. 5 is a schematic diagram of an exemplary system suitable for use with certain aspects disclosed herein.

### DEFINITIONS

In order for the present disclosure to be more readily understood, certain terms are first defined below. Additional definitions for the following terms and other terms may be set forth through the specification. If a definition of a term set forth below is inconsistent with a definition in an application or patent that is incorporated by reference, the definition set forth in this application should be used to understand the meaning of the term.

As used in this specification and the appended claims, the singular forms “a,” “an,” and “the” include plural references unless the context clearly dictates otherwise. Thus, for example, a reference to “a method” includes one or more methods, and/or steps of the type described herein and/or which will become apparent to those persons skilled in the art upon reading this disclosure and so forth.

It is also to be understood that the terminology used herein is for the purpose of describing particular embodiments only, and is not intended to be limiting. Further, unless defined otherwise, all technical and scientific terms used herein have the same meaning as commonly understood by one of ordinary skill in the art to which this disclosure pertains. In describing and claiming the methods, kits, computer readable media, systems, and component parts, the following terminology, and grammatical variants thereof, will be used in accordance with the definitions set forth below.

About: As used herein, “about” or “approximately” or “substantially” as applied to one or more values or elements of interest, refers to a value or element that is similar to a stated reference value or element. In certain embodiments, the term “about” or “approximately” or “substantially” refers to a range of values or elements that falls within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value or element unless otherwise stated or otherwise evident from the context (except where such number would exceed 100% of a possible value or element).

Administer: As used herein, “administer” or “administering” a therapeutic agent or other therapy to a subject means to give, apply or bring the composition into contact with the subject. Administration can be accomplished by any of a number of routes, including, for example, topical, oral, subcutaneous, intramuscular, intraperitoneal, intravenous, intrathecal and intradermal.

Detect: As used herein, “detect,” “detecting,” or “detection” refers to an act of determining the existence or presence of one or more pathologies, or properties indicative thereof, in a subject.

Ear Pathology Model: As used herein, “ear pathology model” refers to a computer algorithm or implementing system that performs otological detections, diagnoses, decision-making, and/or related tasks that typically rely solely on expert human intelligence (e.g., an otolaryngologist or the like). In some embodiments, an ear pathology model is produced using reference otological images and/or videos as training data, which is used to train a machine learning algorithm or other artificial intelligence-based application.

Hyperspectral Imaging: As used herein, “hyperspectral imaging” or “HSI” refers to a technique that evaluates a broad spectrum of electromagnetic radiation in lieu of simply assigning primary colors (red, green, and blue) to each pixel in a given image. Instead, in HSI, light striking each pixel is typically broken down into many different spectral bands in order to provide additional information regarding the image under consideration.

Indexed: As used herein, “indexed” refers to a first element (e.g., clinical information) linked to a second element (e.g., a given sample, a given subject, a recommended therapy, etc.).

Machine Learning Algorithm: As used herein, “machine learning algorithm” generally refers to an algorithm, executed by computer, that automates analytical model building, e.g., for clustering, classification or pattern recognition. Machine learning algorithms may be supervised or unsupervised. Learning algorithms include, for example, artificial neural networks (e.g., back propagation networks), discriminant analyses (e.g., Bayesian classifier or Fisher's analysis), support vector machines, decision trees (e.g., recursive partitioning processes such as CART—classification and regression trees, or random forests), linear classifiers (e.g., multiple linear regression (MLR), partial least squares (PLS) regression, and principal components regression), hierarchical clustering, and cluster analysis. A dataset on which a machine learning algorithm learns can be referred to as “training data.” A model produced using a machine learning algorithm is generally referred to herein as a “machine learning model.”

Match: As used herein, “match” means that at least a first value or element is at least approximately equal to at least a second value or element. In certain embodiments, for example, one or more properties of a captured image (e.g., patterns or the like within the image) from a test subject are used to detect a pathology in the test subject when those properties are at least approximately equal to one or more properties of an ear pathology model.

Pathology: As used herein, “pathology” refers to a deviation from a normal state of health, such as a disease, abnormal condition, or disorder.

Reference Images: As used herein, “reference images” or “reference videos” refer a set of images and/or videos (e.g., a sequence of images) having or known to have or lack specific properties (e.g., known pathologies in associated subjects and/or the like) that is used to generate ear pathology models (e.g., as training data) and/or analyzed along with or compared to test images and/or videos in order to evaluate the accuracy of an analytical procedure. A set of reference images typically includes from at least about 25 to at least about 10,000,000 or more reference images and/or videos. In some embodiments, a set of reference images and/or videos includes about 50, 75, 100, 150, 200, 300, 400, 500, 600, 700, 800, 900, 1,000, 2,500, 5,000, 7,500, 10,000, 15,000, 20,000, 25,000, 50,000, 100,000, 1,000,000, or more reference images and/or videos.

Subject: As used herein, “subject” or “test subject” refers to an animal, such as a mammalian species (e.g., human) or avian (e.g., bird) species. More specifically, a subject can be a vertebrate, e.g., a mammal such as a mouse, a primate, a simian or a human. Animals include farm animals (e.g., production cattle, dairy cattle, poultry, horses, pigs, and the like), sport animals, and companion animals (e.g., pets or support animals). A subject can be a healthy individual, an individual that has or is suspected of having a disease or pathology or a predisposition to the disease or pathology, or an individual that is in need of therapy or suspected of needing therapy. The terms “individual” or “patient” are intended to be interchangeable with “subject.” A “reference subject” refers to a subject known to have or lack specific properties (e.g., known otologic or other pathology and/or the like).

### DETAILED DESCRIPTION

With nine million children affected per year, ear infections are a leading diagnosis for acute care visits in the U.S., and the most common reason children receive antibiotics. However, healthcare providers often have uncertainty in identifying ear infections, which leads to over-prescribing of antibiotics and subsequent antibiotic-resistant bacteria. Up to 26% of antibiotics prescribed for ear infections are not necessary [Soni A. Statistical Brief: #228. Ear Infections (Otitis Media) in Children (0-17): Use and Expenditures, 2006. Medical Expenditure Panel Survey. 2008:1-5]. Uncertainty of the ear exam stems from its small, complex anatomy, a brief glimpse in a moving child, and the design of traditional otoscopes that makes learning and mastering the exam challenging.

To address the limitations of the pre-existing technology, the present disclosure provides an artificial intelligence (AI)-based digital otoscope of use in diagnosing and managing ear infections in certain embodiments. In some implementations, the present disclosure also relates to mobile applications (apps) that feature image recognition using machine learning algorithms to give a diagnosis, or at least an AI augmented diagnosis, of the ear exam and provide management recommendations to healthcare providers and other users. A digital image of the ear exam, aided by the diagnosis provided by the mobile app, improves provider certainty of the diagnosis and proper use of antibiotics for ear infections, among other attributes. In some embodiments, the present disclosure provides ergonomic otoscope devices that are configured for real-time digital image capture and data analysis in addition to having connectivity (e.g., wireless connectivity) to patients' electronic medical records (EMRs) (e.g., Epic electronic health record (EHR) system, etc.). In certain of these embodiments, the smart otoscope devices disclosed herein also include functional side-ports or channels for instruments to clean wax or retrieve foreign bodies during an examination. These devices enable users, irrespective of their level of training or experience, the ability to identify and treat ear infections or other pathologies with the precision of an ear specialist (otologist) and to otherwise improve diagnostic accuracy and otologic disease management.

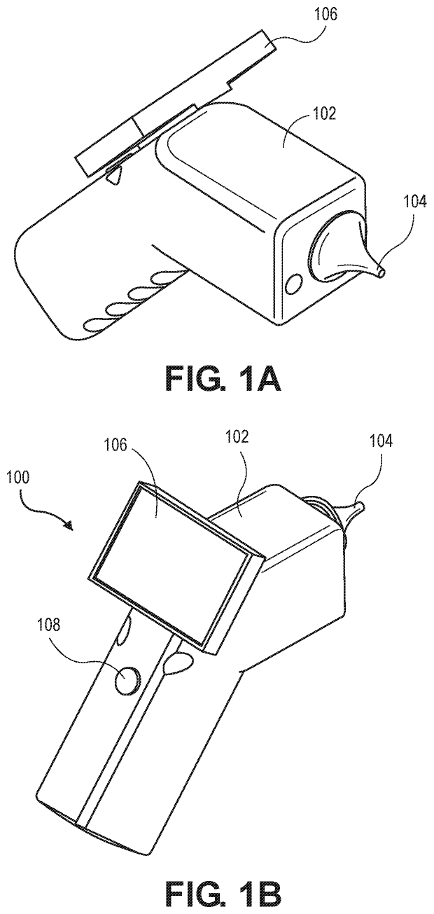

To illustrate, FIGS. 1A and B schematically depict an otoscope device from perspective views according to one exemplary embodiment. As shown, otoscope device 100 includes body structure 102 and disposable speculum 104 removably attached to body structure 102. Although not within view, a camera (e.g., a high-definition (HD) endoscopic camera or the like) is partially disposed within speculum 104 and body structure 102. The camera is configured to capture images and/or videos of an ear canal and/or tympanic membrane of an ear of a subject when the speculum is disposed at least proximal to (e.g., inserted into) the ear canal of a subject. In some embodiments, cameras include microscopes and/or miniature cameras to further magnify ear canals and tympanic membranes as images and/or videos are captured. Otoscope device 100 also typically includes one or more illumination sources (e.g., strobe light emitting diodes (LEDs) or the like) that illuminate the ear canal and tympanic membrane of an ear of a subject when the images and/or videos are captured using the camera to improve image quality. Otoscope device 100 also includes display screen 106 (e.g., a liquid crystal display (LCD) screen or the like) connected to body structure 102. Display screen 106 is configured to display the images and/or videos of the ear canal and/or tympanic membrane of the ear of a subject during an examination procedure. Otoscope device 100 also includes control button 108, which is used by a user to control operation of the device, including capturing images and/or videos.

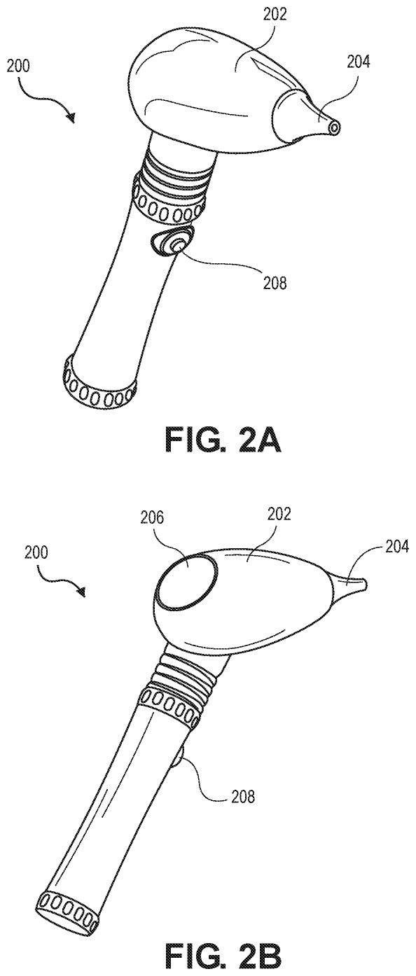

As a further exemplary illustration, FIGS. 2A-C schematically depict an otoscope device from various views according to one embodiment. As shown, otoscope device 200 includes body structure 202 and disposable speculum 204 removably attached to body structure 202. A camera (not within view) is partially disposed within speculum 204 and body structure 202. The camera is used to capture images and/or videos of a subject's ear canal and tympanic membrane during an examination process. Otoscope device 200 also generally includes at least one illumination source that illuminates the ear canal and tympanic membrane of an ear of a subject to improve image quality when the images and/or videos are captured using the camera. Illumination sources are described further herein. As also shown, otoscope device 200 also includes display screen 206 (e.g., a liquid crystal display (LCD) screen or the like) connected to body structure 202. Display screen 206 displays the images and/or videos of the ear canal and/or tympanic membrane of the ear of a subject during an examination procedure. Otoscope device 200 also includes control button 208, which is used by a user to control operation of the device, including to selectively capture images and/or videos.

In some embodiments, a device body structure and/or speculum includes a working port or channel through which an implement is inserted into the ear canal of a subject during an examination procedure. The implement is typically used to clean, retrieve foreign bodies from, and/or take samples from the ear canal and/or the tympanic membrane of the ear of the subject.

The otoscope devices disclosed herein, including otoscope devices 100 and 200, also generally include a controller (e.g., a local processor, etc.) at least partially disposed within the device body structures. A controller is general operably connected the camera (e.g., disposed within the camera structure in certain embodiments) and to the display screen. In some embodiments, the controller is configured to capture images and/or videos using hyperspectral imaging. In addition, the controller typically includes, or is capable of accessing (e.g., remotely via a wireless connection), computer readable media (e.g., embodying an artificial intelligence (AI)-based algorithm) comprising non-transitory computer executable instructions which, when executed by at least one electronic processor, perform capturing images and/or videos of the ear canal and/or tympanic membrane of the ear of a subject, and displaying the captured images and/or videos on the display screen. The computer executable instructions also perform matching one or more properties (e.g., test pixel or other image patterns) of the captured images and/or videos with one or more properties e.g., reference pixel or other image patterns) of an ear pathology model that is trained on a plurality of reference images and/or videos (e.g., about 50, about 100, about 500, about 1,000, about 10,000, or more reference images and/or videos) of ear canals and/or tympanic membranes of ears of reference subjects. The properties of the ear pathology model are typically indicative of at least one ear-related pathology (e.g., otitis media, otosclerosis, keratosis obturans, tympanosclerosis, etc.). The ear pathology models disclosed herein are typically generated using one or more machine learning algorithms. In some of these embodiments, the machine learning algorithms include one or more neural networks. In certain embodiments, ear pathology models include selected therapies indexed to a given otologic pathology to provide therapy recommendations to healthcare providers or other users when the pathology is detected in a subject.

The controllers of the otoscope devices disclosed herein include various embodiments. In some embodiments, for example, the controller of a given device is wirelessly connected, or connectable, to one or more of the computer executable instructions. In certain embodiments, the controller is operably connected, or connectable, to a database that includes electronic medical records (EMRs) of subjects. In these embodiments, the computer executable instructions typically further perform retrieving data from the electronic medical record and/or populating the electronic medical record with at least one of the images and/or videos, selected smart phrases, and/or other related information. In certain of these embodiments, the controller is wirelessly connected, or connectable, to the electronic medical records. Typically, the otoscope device and/or the database is wirelessly connected, or connectable, to one or more communication devices (e.g., mobile phones, tablet computers, etc.) of remote users. This enables the remote users to view the captured images and/or videos of the ear canal and/or tympanic membrane of the ear of a given subject and/or the electronic medical record of that subject using the communication devices. In some of these embodiments, the communication devices include one or more mobile applications that operably interface with the otoscope device and/or the database. In these embodiments, the remote users are generally capable of inputting entries into the electronic medical record of the subject in view of a detected pathology in the ear of the subject using the communication devices. In some of these embodiments, the users are capable of ordering one or more therapies and/or additional analyses of the subject in view of the detected pathology in the ear of the subject using the communication devices.

The otoscope devices disclosed herein typically include an illumination source (e.g., a strobe LED or the like) operably connected to the controller. The illumination source is typically configured to illuminate the ear canal and/or tympanic membrane of an ear of the subject when the speculum is disposed at least proximal to the ear canal and/or tympanic membrane of the ear of a given subject. In some embodiments, the illumination source is configured to illuminate at two or more selectable illumination wavelengths (e.g., at least one visible wavelength and/or at least one infrared wavelength). In these embodiments, the computer executable instructions typically further perform causing the illumination source to illuminate at a selected illumination wavelength in response to a user selection. In some embodiments, the illumination source is configured to illuminate in one or more selectable illumination modes (e.g., a pulsed illumination mode, an illumination intensity, etc.). In these embodiments, the computer executable instructions generally further perform causing the illumination source to illuminate at a selected illumination mode in response to a user selection.

The otoscope devices disclosed herein also include power sources operably connected, or connectable, to controllers, cameras, and/or display screens. Essentially any power source is optionally adapted for use with the otoscope devices. In some embodiments, the power source is a rechargeable battery, whereas in other embodiments, the power source is an external electricity outlet to which a given otoscope device is connected via a power cord.

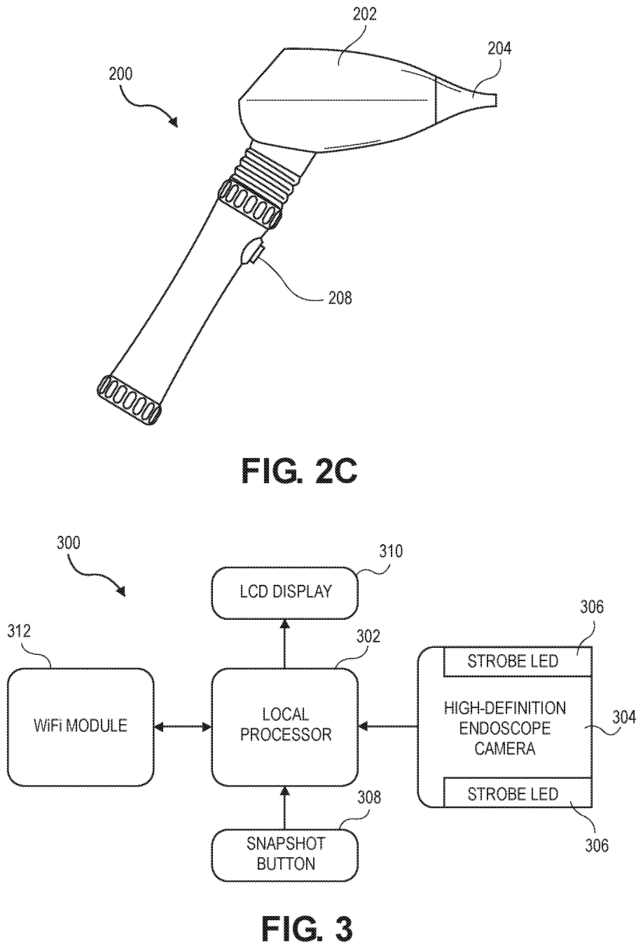

As a further illustration, FIG. 3 is a schematic diagram that depicts exemplary otoscope device components according to some aspects disclosed herein. As shown, otoscope device 300 includes controller 302 (shown as a local processor disposed within the device). During device operation, controller 302 receives captured images and/or videos from high-definition (HD) endoscopic camera 304, which is associated with strobe LEDs 306. Strobe LEDs 306 are used to illuminate the ear canal of a subject as images and/or videos are captured by HD endoscopic camera 304 during an examination process to improve image quality. A user selectively engages (e.g., presses) snapshot button 308, which is operably connected to controller 302, to effect image capture. The captured images and/or videos are displayed on LCD display 310, which is also operably connected to controller 302. As also shown, otoscope device 300 also includes a wireless connectivity module (Wifi module) 312 that operably interfaces with controller 302. Wireless connectivity module 312 is configured to interface with remote databases (e.g., electronic medical records, reference image data sets, etc.), communication devices (e.g., mobile phones, tablet computers, notebook computers, etc.), computer readable media (e.g., ear pathology models, pattern recognition software, etc.), and/or the like.

In some embodiments, the otoscope devices of the present disclosure are provided as components of kits. Various kit configurations are optionally utilized, but in certain embodiments, one or more otoscope devices are packaged together with computer readable media, replacement specula, replacement illumination sources (e.g., LEDs, etc.), rechargeable battery charging stations, batteries, operational instructions, and/or the like.

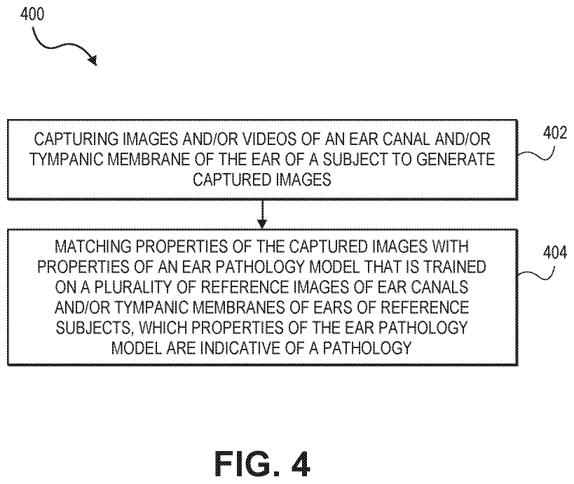

FIG. 4 is a flow chart that schematically depicts exemplary method steps of detecting an otologic pathology according to some aspects disclosed herein. As shown, method 400 includes capturing (using an otoscope device, as disclosed herein) images and/or videos of an ear canal and/or tympanic membrane of the ear of a subject (step 402). In some embodiments, method 400 includes using hyperspectral imaging to capture the images and/or videos of the ear canal and/or tympanic membrane of the ear of the subject. Method 400 also includes matching properties (e.g., image/pixel patterns, etc.) of the captured images and/or videos with properties (e.g., image/pixel patterns, etc.) of an ear pathology model (step 404). The ear pathology model is trained on a plurality of reference images and/or videos (e.g., about 50, about 100, about 500, about 1,000, about 10,000, or more reference images and/or videos) of ear canals and/or tympanic membranes of ears of reference subjects. The properties of the ear pathology model are generally indicative of the given otologic pathology. Typically, steps 402 and 404 are performed substantially in real-time during a given examination procedure.

In some embodiments, method 400 is repeated at one or more later time points to monitor progression of the pathology in the subject. In certain embodiments, method 400 includes administering one or more therapies to the subject to treat the pathology. In some of these embodiments, remote users (e.g., healthcare providers) order the therapies and/or additional analyses of the subject in view of the detected pathology in the ear of the subject using a communication device, such as a mobile phone or remote computing system. In certain of these embodiments, a system that comprises the database automatically orders the therapies and/or additional analyses of the subject in view of the detected pathology in the ear of the subject when remote users input the entries into the electronic medical record of the subject. Additional aspects of methods of using otoscope devices are described herein.

Essentially any otologic or ear-related pathology can be detected and diagnosed using the otoscope device disclosed herein. Examples of such pathologies, include otitis media, otitis media with effusion, mucoid otitis media, otosclerosis, cholesteatoma, direct trauma, infected furuncle, necrotizing otitis externa, herpes zoster, acquired stenosis, osteoma, acute otitis externa, chronic otitis externa, deep impacted wax, retraction pocket, keratosis obturans, granular myringitis, bullous myringitis, tympanosclerosis, perforation, tympanosclerosis, glomus tumour, posterior infection, hemotympanum, foreign body, and temporal bone fracture, among other pathologies.

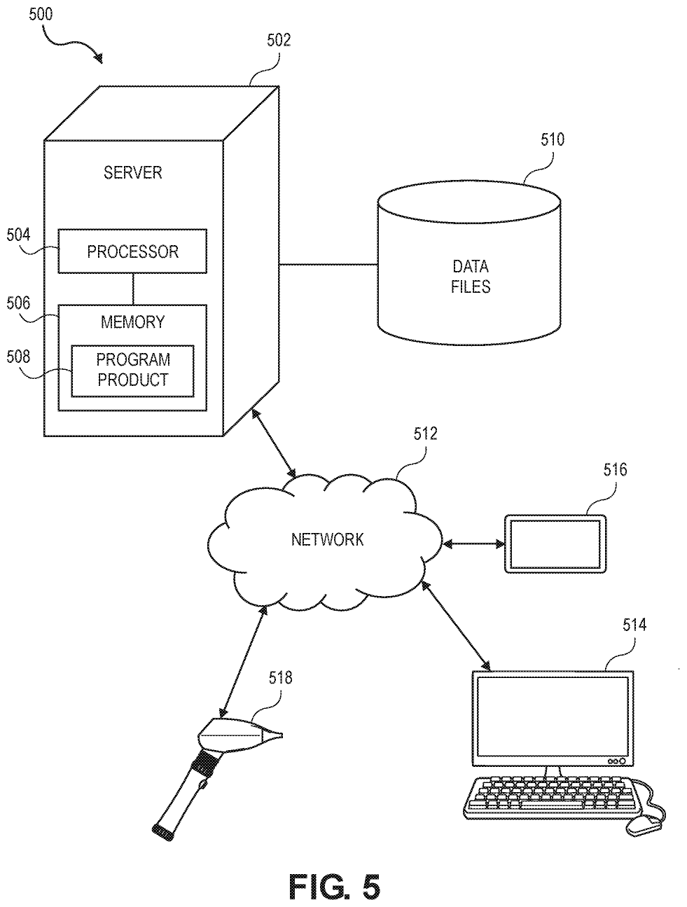

The present disclosure also provides various systems and computer program products or machine readable media. In some aspects, for example, the methods described herein are optionally performed or facilitated at least in part using systems, distributed computing hardware and applications (e.g., cloud computing services), electronic communication networks, communication interfaces, computer program products, machine readable media, electronic storage media, software (e.g., machine-executable code or logic instructions) and/or the like. To illustrate, FIG. 5 provides a schematic diagram of an exemplary system suitable for use with implementing at least aspects of the methods disclosed in this application. As shown, system 500 includes at least one controller or computer, e.g., server 502 (e.g., a search engine server), which includes processor 504 and memory, storage device, or memory component 506, and one or more other communication devices 514, 516, (e.g., client-side computer terminals, telephones, tablets, laptops, other mobile devices, etc. (e.g., for receiving captured images and/or videos for further analysis, etc.)) positioned remote from otoscope device 518, and in communication with the remote server 502, through electronic communication network 512, such as the Internet or other internetwork. Communication devices 514, 516 typically include an electronic display (e.g., an internet enabled computer or the like) in communication with, e.g., server 502 computer over network 512 in which the electronic display comprises a user interface (e.g., a graphical user interface (GUI), a web-based user interface, and/or the like) for displaying results upon implementing the methods described herein. In certain aspects, communication networks also encompass the physical transfer of data from one location to another, for example, using a hard drive, thumb drive, or other data storage mechanism. System 500 also includes program product 508 (e.g., related to an ear pathology model) stored on a computer or machine readable medium, such as, for example, one or more of various types of memory, such as memory 506 of server 502, that is readable by the server 502, to facilitate, for example, a guided search application or other executable by one or more other communication devices, such as 514 (schematically shown as a desktop or personal computer). In some aspects, system 500 optionally also includes at least one database server, such as, for example, server 510 associated with an online website having data stored thereon (e.g., entries corresponding to more reference images and/or videos, indexed therapies, etc.) searchable either directly or through search engine server 502. System 500 optionally also includes one or more other servers positioned remotely from server 502, each of which are optionally associated with one or more database servers 510 located remotely or located local to each of the other servers. The other servers can beneficially provide service to geographically remote users and enhance geographically distributed operations.

As understood by those of ordinary skill in the art, memory 506 of the server 502 optionally includes volatile and/or nonvolatile memory including, for example, RAM, ROM, and magnetic or optical disks, among others. It is also understood by those of ordinary skill in the art that although illustrated as a single server, the illustrated configuration of server 502 is given only by way of example and that other types of servers or computers configured according to various other methodologies or architectures can also be used. Server 502 shown schematically in FIG. 5, represents a server or server cluster or server farm and is not limited to any individual physical server. The server site may be deployed as a server farm or server cluster managed by a server hosting provider. The number of servers and their architecture and configuration may be increased based on usage, demand and capacity requirements for the system 500. As also understood by those of ordinary skill in the art, other user communication devices 514, 516 in these aspects, for example, can be a laptop, desktop, tablet, personal digital assistant (PDA), cell phone, server, or other types of computers. As known and understood by those of ordinary skill in the art, network 512 can include an internet, intranet, a telecommunication network, an extranet, or world wide web of a plurality of computers/servers in communication with one or more other computers through a communication network, and/or portions of a local or other area network.

As further understood by those of ordinary skill in the art, exemplary program product or machine readable medium 508 is optionally in the form of microcode, programs, cloud computing format, routines, and/or symbolic languages that provide one or more sets of ordered operations that control the functioning of the hardware and direct its operation. Program product 508, according to an exemplary aspect, also need not reside in its entirety in volatile memory, but can be selectively loaded, as necessary, according to various methodologies as known and understood by those of ordinary skill in the art.

As further understood by those of ordinary skill in the art, the term “computer-readable medium” or “machine-readable medium” refers to any medium that participates in providing instructions to a processor for execution. To illustrate, the term “computer-readable medium” or “machine-readable medium” encompasses distribution media, cloud computing formats, intermediate storage media, execution memory of a computer, and any other medium or device capable of storing program product 508 implementing the functionality or processes of various aspects of the present disclosure, for example, for reading by a computer. A “computer-readable medium” or “machine-readable medium” may take many forms, including but not limited to, non-volatile media, volatile media, and transmission media. Non-volatile media includes, for example, optical or magnetic disks. Volatile media includes dynamic memory, such as the main memory of a given system. Transmission media includes coaxial cables, copper wire and fiber optics, including the wires that comprise a bus. Transmission media can also take the form of acoustic or light waves, such as those generated during radio wave and infrared data communications, among others. Exemplary forms of computer-readable media include a floppy disk, a flexible disk, hard disk, magnetic tape, a flash drive, or any other magnetic medium, a CD-ROM, any other optical medium, punch cards, paper tape, any other physical medium with patterns of holes, a RAM, a PROM, and EPROM, a FLASH-EPROM, any other memory chip or cartridge, a carrier wave, or any other medium from which a computer can read.

Program product 508 is optionally copied from the computer-readable medium to a hard disk or a similar intermediate storage medium. When program product 508, or portions thereof, are to be run, it is optionally loaded from their distribution medium, their intermediate storage medium, or the like into the execution memory of one or more computers, configuring the computer(s) to act in accordance with the functionality or method of various aspects. All such operations are well known to those of ordinary skill in the art of, for example, computer systems.

To further illustrate, in certain aspects, this application provides systems that include one or more processors, and one or more memory components in communication with the processor. The memory component typically includes one or more instructions that, when executed, cause the processor to provide information that causes at least one captured image, EMR, and/or the like to be displayed (e.g., via otoscope 518 and/or via communication devices 514, 516 or the like) and/or receive information from other system components and/or from a system user (e.g., via otoscope 518 and/or via communication devices 514, 516, or the like).

In some aspects, program product 508 includes non-transitory computer-executable instructions which, when executed by electronic processor 504 perform at least: capturing images and/or videos of the ear canal and/or tympanic membrane of the ear of a subject when the speculum is disposed at least proximal to the ear canal and/or tympanic membrane of the ear of the subject, and matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of at least one pathology. Other exemplary executable instructions that are optionally performed are described further herein.

Additional details relating to computer systems and networks, databases, and computer program products are also provided in, for example, Peterson, Computer Networks: A Systems Approach, Morgan Kaufmann, 5th Ed. (2011), Kurose, Computer Networking: A Top-Down Approach, Pearson, 7^{th} Ed. (2016), Elmasri, Fundamentals of Database Systems, Addison Wesley, 6th Ed. (2010), Coronel, Database Systems: Design, Implementation, & Management, Cengage Learning, 11^{th} Ed. (2014), Tucker, Programming Languages, McGraw-Hill Science/Engineering/Math, 2nd Ed. (2006), and Rhoton, Cloud Computing Architected: Solution Design Handbook, Recursive Press (2011), which are each incorporated by reference in their entirety.

While the foregoing disclosure has been described in some detail by way of illustration and example for purposes of clarity and understanding, it will be clear to one of ordinary skill in the art from a reading of this disclosure that various changes in form and detail can be made without departing from the true scope of the disclosure and may be practiced within the scope of the appended claims. For example, all the methods, devices, systems, computer readable media, and/or component parts or other aspects thereof can be used in various combinations. All patents, patent applications, websites, other publications or documents, and the like cited herein are incorporated by reference in their entirety for all purposes to the same extent as if each individual item were specifically and individually indicated to be so incorporated by reference.

1. A method of detecting a pathology in an ear of a subject, the method comprising:
capturing, by an otoscope, one or more images and/or videos of an ear canal and/or tympanic membrane of the ear of the subject, wherein the otoscope comprises at least one camera to generate at least one captured image and/or video; and,
matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of the pathology, thereby detecting the pathology in the ear of the subject.

2. (canceled)

3. The method of claim 1, wherein the properties comprise one or more patterns.

4. The method of claim 1, wherein the ear pathology model is generated using one or more machine learning algorithms.

5. (canceled)

6. The method of claim 1, wherein the capturing and matching steps are performed substantially in real-time.

7. The method of claim 1, wherein the pathology comprises one or more of: otitis media, otitis media with effusion, mucoid otitis media, otosclerosis, cholesteatoma, direct trauma, infected furuncle, necrotizing otitis externa, herpes zoster, acquired stenosis, osteoma, acute otitis externa, chronic otitis externa, deep impacted wax, retraction pocket, keratosis obturans, granular myringitis, bullous myringitis, tympanosclerosis, perforation, tympanosclerosis, glomus tumour, posterior infection, hemotympanum, foreign body, and temporal bone fracture.

8. The method of claim 1, further comprising administering one or more therapies to the subject to treat the pathology.

9. (canceled)

10. (canceled)

11. The method of claim 1, wherein the otoscope comprises one or more working ports or channels and the method further comprises inserting one or more implements through the working ports or channels into the ear canal of the subject to clean, retrieve foreign bodies from, and/or take samples from the ear canal and/or the tympanic membrane of the ear of the subject when the otoscope captures the images and/or videos of the ear canal and/or the tympanic membrane of the ear of the subject.

12. (canceled)

13. The method of claim 1, comprising using hyperspectral imaging and/or optical coherence tomography (OCT) to capture the images and/or videos of the ear canal and/or tympanic membrane of the ear of the subject.

14. The method of claim 1, wherein the otoscope is operably connected to a database comprising an electronic medical record of the subject and wherein the method further comprises retrieving data from the electronic medical record and/or populating the electronic medical record with at least one of the images and/or videos, and/or information related thereto.

15-20. (canceled)

21. The method of claim 1, wherein the otoscope comprises at least one illumination source and the method further comprises illuminating the ear canal and/or tympanic membrane of the ear of the subject using the illumination source when capturing the images and/or videos of the ear canal and/or tympanic membrane of the ear of the subject.

22-26. (canceled)

27. An otoscope device, comprising:
a body structure;
at least one speculum operably connected to the body structure;
at least one camera at least partially disposed within the speculum, which camera is configured to capture one or more images and/or videos of an ear canal and/or tympanic membrane of an ear of a subject when the speculum is disposed at least proximal to the ear canal and/or tympanic membrane of the ear of the subject;
at least one display screen operably connected to the body structure, which display screen is configured to display the images and/or videos of the ear canal and/or tympanic membrane of the ear of the subject when the speculum is disposed at least proximal to the ear canal and/or tympanic membrane of the ear of the subject;
at least one controller at least partially disposed within the body structure, which controller is operably connected at least to the camera and to the display screen, wherein the controller comprises, or is capable of accessing, computer readable media comprising non-transitory computer executable instructions which, when executed by at least one electronic processor, perform at least:
capturing the images and/or videos of the ear canal and/or tympanic membrane of the ear of the subject when the speculum is disposed at least proximal to the ear canal and/or tympanic membrane of the ear of the subject;
displaying the captured images and/or videos of the ear canal and/or tympanic membrane of the ear of the subject on the display screen at least when the speculum is disposed at least proximal to the ear canal and/or tympanic membrane of the ear of the subject; and
matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of at least one pathology; and,
at least one power source operably connected, or connectable, to one or more of the controller, the camera, and the display screen.

28. A kit comprising the otoscope device of claim 27.

29. (canceled)

30. The otoscope device of claim 27, wherein the properties comprise one or more patterns.

31. The otoscope device of claim 27, wherein the ear pathology model is generated using one or more machine learning algorithms.

32. (canceled)

33. The otoscope device of claim 27, wherein the ear pathology model comprises one or more selected therapies indexed to the pathology.

34. The otoscope device of claim 27, wherein the body structure and/or the speculum comprises one or more working ports or channels through which one or more implements are inserted into the ear canal of the subject to clean, retrieve foreign bodies from, and/or take samples from the ear canal and/or the tympanic membrane of the ear of the subject when the otoscope captures the images and/or videos of the ear canal and/or the tympanic membrane of the ear of the subject.

35. (canceled)

36. The otoscope device of claim 27, wherein the controller is operably connected, or connectable, to a database comprising an electronic medical record of the subject and wherein the computer executable instructions further perform retrieving data from the electronic medical record and/or populating the electronic medical record with at least one of the images and/or videos, and/or information related thereto.

37-41. (canceled)

42. The otoscope device of claim 27, wherein the controller is configured to capture the images and/or videos of the ear canal and/or tympanic membrane of the ear of the subject using hyperspectral imaging and/or optical coherence tomography (OCT).

43. The otoscope device of claim 27, further comprising at least one illumination source operably connected to the controller, which illumination source is configured to illuminate the ear canal and/or tympanic membrane of an ear of the subject when the speculum is disposed at least proximal to the ear canal and/or tympanic membrane of the ear of the subject.

44-47. (canceled)

48. A system, comprising:
at least one otoscope device that comprises at least one camera that is configured to capture one or more images and/or videos of an ear canal and/or tympanic membrane of an ear of a subject when the camera is disposed at least proximal to the ear canal and/or tympanic membrane of the ear of the subject;
at least one controller that is operably connected, or connectable, at least to the camera, wherein the controller comprises, or is capable of accessing, computer readable media comprising non-transitory computer executable instructions which, when executed by at least one electronic processor, perform at least:
capturing the images and/or videos of the ear canal and/or tympanic membrane of the ear of the subject when the speculum is disposed at least proximal to the ear canal and/or tympanic membrane of the ear of the subject; and,
matching one or more properties of the captured images and/or videos with one or more properties of at least one ear pathology model that is trained on a plurality of reference images and/or videos of ear canals and/or tympanic membranes of ears of reference subjects, which properties of the ear pathology model are indicative of at least one pathology.

49. (canceled)
